(19) United States
(12) Patent Application Publication
(10) Pub. No.: US 2022/0409431 A1
(43) Pub. Date: Dec. 29, 2022

(54) COMPACT VALVE

(71) Applicant: JOHNSON & JOHNSON SURGICAL VISION, INC., Santa Ana, CA (US)

(72) Inventors: Yehuda Algawi, Binyamina (IL); Assaf Govari, Haifa (IL); Eran Aharon, Haifa (IL); Ilya Sitnitsky, Nahariya (IL)

(21) Appl. No.: 17/355,884

(22) Filed: Jun. 23, 2021

(51) Int. Cl. A61F 9/007; A61M 1/00

(52) U.S. Cl. CPC A61F 9/00745; A61M 1/774; A61M 1/743; A61M 2205/8287; A61M 2210/0612

(57) ABSTRACT

In one embodiment, a fluid dynamics system includes a solenoid valve including a valve body, which includes a valve cavity having a direction of elongation, a first channel, and a second channel, a solenoid coil disposed in the valve body around the valve cavity, and a plunger comprising a magnetic element and configured to move back-and-forth along the direction of elongation between a first position and a second position in the valve cavity, selectively opening the first channel and closing the second channel when the plunger is in the first position, and closing the first channel and opening the second channel when the plunger is in the second position, and a controller configured to control the solenoid coil to selectively move the plunger between the first position and the second position, and to selectively maintain the plunger in the first position and the second position.

### FIELD OF THE INVENTION

The present invention relates to medical systems, and in particular, but not exclusively to, fluid dynamics in medical systems.

### BACKGROUND

A cataract is a clouding and hardening of the eye's natural lens, a structure which is positioned behind the cornea, iris and pupil. The lens is mostly made up of water and protein and as people age these proteins change and may begin to clump together obscuring portions of the lens. To correct this, a physician may recommend phacoemulsification cataract surgery. In the procedure, the surgeon makes a small incision in the sclera or cornea of the eye. Then a portion of the anterior surface of the lens capsule is removed to gain access to the cataract. The surgeon then uses a phacoemulsification probe, which has an ultrasonic handpiece with a needle. The tip of the needle vibrates at ultrasonic frequency to sculpt and emulsify the cataract while a pump aspirates particles and fluid from the eye through the tip. Aspirated fluids are replaced with irrigation of a balanced salt solution (BSS) to maintain the anterior chamber of the eye. After removing the cataract with phacoemulsification, the softer outer lens cortex is removed with suction. An intraocular lens (IOL) is then introduced into the empty lens capsule restoring the patient's vision.

### SUMMARY

There is provided in accordance with an embodiment of the present disclosure, a fluid dynamics system, including a solenoid valve including a valve body including a valve cavity having a direction of elongation, a first channel, and a second channel, a solenoid coil disposed in the valve body around the valve cavity, and a plunger including a magnetic element and configured to move back-and-forth along the direction of elongation between a first position and a second position in the valve cavity, selectively opening the first channel and closing the second channel when the plunger is in the first position, and closing the first channel and opening the second channel when the plunger is in the second position, and a controller configured to control the solenoid coil to selectively move the plunger between the first position and the second position, and to selectively maintain the plunger in the first position and the second position.

Further in accordance with an embodiment of the present disclosure the plunger does not have a fixed rest position in the valve cavity.

Still further in accordance with an embodiment of the present disclosure the plunger does not include a restoring element configured to restore the plunger to a fixed rest position.

Additionally, in accordance with an embodiment of the present disclosure the plunger will not remain in the first position and the second position without applying a current to the solenoid coil.

Moreover, in accordance with an embodiment of the present disclosure the plunger will remain in the first position or the second position upon application of a current to the solenoid coil.

Further in accordance with an embodiment of the present disclosure the solenoid coil has a center with respect to the direction of elongation, the first position and the second position are on either side of the center of the solenoid coil with respect to the direction of elongation, and the controller is configured to change a polarity of the solenoid coil from a first polarity to a second polarity and then back to the first polarity to move the plunger from the first position to the second position, and change the polarity of the solenoid coil from the first polarity to the second polarity and then back to the first polarity to move the plunger from the second position to the first position.

Still further in accordance with an embodiment of the present disclosure the controller is configured to control the solenoid coil to maintain the first polarity in order to maintain the plunger in position.

Additionally, in accordance with an embodiment of the present disclosure the magnetic element has a center with respect to the direction of elongation, and the controller is configured to change the polarity from the second polarity to the first polarity responsively to the center of the magnetic element passing the center of the solenoid coil with respect to the direction of elongation.

Moreover, in accordance with an embodiment of the present disclosure the solenoid valve includes a sensor configured to provide a signal responsively to a relative position of the center of the magnetic element to the center of the solenoid coil with respect to the direction of elongation, and the controller is configured to change the polarity from the second polarity to the first polarity responsively to the provided signal.

Further in accordance with an embodiment of the present disclosure the sensor includes a Hall-effect sensor.

Still further in accordance with an embodiment of the present disclosure, the system includes a phacoemulsification probe including a distal end including a needle, an irrigation channel configured to convey irrigation fluid to the distal end, an aspiration channel configured to convey eye fluid and waste matter away from the distal end, the aspiration channel including the first channel, and a bypass channel connected to the irrigation channel and the aspiration channel, the bypass channel including the second channel, and wherein the controller is configured to selectively control the solenoid coil to (a) move the plunger to the first position to open the first channel of the aspiration channel and close the second channel of the bypass channel allowing aspiration of the eye fluid and waste matter away from the distal end, and (b) move the plunger to the second position to close the first channel of the aspiration channel and open the second channel of the bypass channel allowing a portion of the irrigation fluid in the irrigation channel to enter the aspiration channel reducing a vacuum in part of the aspiration channel.

Additionally in accordance with an embodiment of the present disclosure, the system includes an aspiration tubing line, and a pumping sub-system configured to be coupled to the aspiration tubing line and pump the eye fluid and waste matter away from the distal end via the aspiration tubing line and the aspiration channel, and wherein the first channel of the aspiration channel includes a first section connected to the distal end, and a second section configured to be coupled to the pumping sub-system via the aspiration tubing line, the bypass channel being configured to allow the portion of the irrigation fluid in the irrigation channel to enter the second section of the aspiration channel when the plunger is in the second position.

Moreover, in accordance with an embodiment of the present disclosure, the system includes a sensor configured to provide a signal indicative of a fluid metric in the second section of the aspiration channel, and wherein the controller is configured to control the solenoid coil to selectively move the plunger between the first position and the second position responsively to the signal.

Further in accordance with an embodiment of the present disclosure the fluid metric is a pressure level,

Still further in accordance with an embodiment of the present disclosure the controller is configured to detect a rate of change of the fluid metric in the second section of the aspiration channel, and control the solenoid coil to move the plunger to the second position responsively to the detected rate of change passing a given rate of change allowing the portion of the irrigation fluid in the irrigation channel to enter the second section of the aspiration channel via the bypass channel increasing the fluid metric in the second section of the aspiration channel.

Additionally, in accordance with an embodiment of the present disclosure the controller is configured to control the solenoid coil to move the plunger to the first position responsively to the fluid metric in the second section of the aspiration channel passing a given value.

Moreover, in accordance with an embodiment of the present disclosure the phacoemulsification probe further includes a probe body and a fluid dynamics cartridge configured to be reversibly connected to the probe body, the fluid dynamics cartridge including the solenoid valve, the sensor, and the bypass channel.

There is also provided in accordance with another embodiment of the present disclosure, a fluid dynamics method, including changing a polarity of a solenoid coil from a first polarity to a second polarity and then back to the first polarity to move a plunger including a magnetic element from a first position to a second position in a valve cavity, changing the polarity of the solenoid coil from the first polarity to the second polarity and then back to the first polarity to move the plunger from the second position to the first position, and controlling the solenoid coil to maintain the first polarity in order to maintain the plunger in the first position or the second position.

Further in accordance with an embodiment of the present disclosure the changing the polarity includes changing the polarity of the solenoid coil from the second polarity to the first polarity responsively to a center of the magnetic element passing a center of the solenoid coil with respect to a direction of elongation of the valve cavity.

Still further in accordance with an embodiment of the present disclosure, the method includes providing a signal responsively to a relative position of the center of the magnetic element to the center of the solenoid coil with respect to the direction of elongation, and wherein the changing the polarity is performed responsively to the provided signal.

Additionally, in accordance with an embodiment of the present disclosure the providing is performed by a Hall-effect sensor.

### BRIEF DESCRIPTION OF THE DRAWINGS

The present invention will be understood from the following detailed description, taken in conjunction with the drawings in which:

FIG. 1 is a partly pictorial, partly block diagram view of a phacoemulsification system constructed and operative in accordance with an embodiment of the present invention;

FIGS. 2A-B are views of a probe for use with the system of FIG. 1;

FIG. 3A is a schematic view of an interior of a fluid dynamics cartridge for use in the probe of FIGS. 2A-B;

FIG. 3B is a cross-section of the fluid dynamics cartridge through line B:B of FIG. 3A;

FIG. 3C is a cross-section of the fluid dynamics cartridge through line C:C of FIG. 3A during normal operation;

FIG. 3D is a schematic view illustrating fluid flow through the fluid dynamics cartridge of FIG. 3A during normal operation;

FIG. 3E is a cross-section of the fluid dynamics cartridge through line C:C of FIG. 3A during occlusion release;

FIG. 3F is a schematic view illustrating fluid flow through the fluid dynamics cartridge of FIG. 3A during occlusion release;

FIGS. 4A-G are schematic views illustrating movement of a permanent magnet in a solenoid coil for use in the fluid dynamics cartridge of FIG. 3A;

FIG. 5 is a flowchart including steps in a method of operation of the solenoid coil of FIGS. 4A-G; and

FIG. 6 is a flowchart including steps in a method of operation of system of FIG. 1.

### DESCRIPTION OF EXAMPLE EMBODIMENTS

### Overview

During phacoemulsification of an eye lens, the emulsified lens particles are aspirated. When a particle blocks the inlet of an aspiration channel (which could be in a needle of a phacoemulsification probe) causing occlusion of the channel, the vacuum in the channel increases. When the channel becomes unblocked (e.g., by the particle being subsequently sucked down the channel), the high vacuum in the channel causes an aspiration surge known as post occlusion surge, which may have traumatic consequences to the eye. For example, sensitive parts of the eye may be damaged or could come into contact with the needle of the phacoemulsification probe.

A possible solution to the problem of vacuum level surge is incorporating an aspiration bypass in the probe. Such a bypass may consist of a small hole or channel between an irrigation channel of the probe and the aspiration channel. When a blockage occurs, the high vacuum diverts irrigation fluid into the aspiration channel via the hole, thereby limiting the vacuum level.

However, the above-described bypass aspiration technique is still prone to produce a traumatic aspiration surge when the channel unblocks, since the high vacuum is present in a long tube (which being flexible may also be compressed adding to the vacuum problem) between a portion of the aspiration channel inside the emulsification probe and the aspiration pump, and that large, partially vacant volume, may therefore cause a surge when the occlusion breaks. Moreover, diversion of irrigation fluid may cause an uncontrolled pressure-drop in the irrigation channel, which may also pose a risk to the eye.

Another possible solution to the problem is to provide valves on the aspiration channel and the bypass channel so that when the occlusion breaks the aspiration channel is closed and the bypass channel is opened. In this manner, the occlusion surge is prevented by quickly closing the aspiration channel. The bypass channel is opened to reduce the vacuum in the aspiration channel via irrigation fluid from the irrigation channel entering the aspiration channel via the bypass channel in a non-time critical manner. This possible solution requires having two valves which occupy a lot of space in the probe, which is already crowded with various elements.

Embodiments of the present invention generally solve the above problems by providing a fast-acting, compact, and programmable (optionally four-way, two position) solenoid valve. The solenoid valve includes a solenoid coil (or more than one solenoid coils) which moves a plunger including a magnetic element (such as a permanent magnet) in a valve cavity. The plunger has two main positions in the valve cavity. In one position, a first channel (e.g., an aspiration channel) is closed by the plunger, and a second channel (e.g., a bypass channel connecting an irrigation channel with the aspiration channel) is open. In the other position, the first channel (e.g., aspiration channel) is open, and the second channel (e.g., bypass channel) is closed by the plunger.

In some embodiments, the valve is implemented in a phacoemulsification probe including an aspiration channel with two sections, a first section extending from a distal end of the probe to the valve, and a second section extending from the valve for connection to an aspiration tubing line, which is connected to an aspiration pump for aspirating eye fluid and waste matter away from the distal end of the probe via the aspiration channel and aspiration tubing line. The bypass channel is connected to the second section of the aspiration channel.

In operation, upon detection of an occlusion or occlusion clearance, the valve is actuated quickly (e.g., within 10 milliseconds) to close the aspiration channel, and open the bypass channel so that in a non-time critical manner irrigation fluid enters the second section of the aspiration channel via the bypass channel to reduce the vacuum in the second section of the aspiration channel and the aspiration tubing line. Once the vacuum in the second section of the aspiration channel has sufficiently reduced due to irrigation fluid entering via the bypass channel (e.g., by sensing the pressure/vacuum or waiting a given time period), the aspiration channel may then be reopened allowing fluid to be aspirated without causing a surge.

The solenoid valve does not need a restoring element (such as a spring) to keep the plunger in a rest position when a current is not applied to the solenoid coil. An electric current needs to be applied to the solenoid coil to selectively keep the plunger in the first position or the second position. If a current is not supplied to the solenoid coil, the position of the plunger may be unstable and unknown. Using a solenoid valve without a restoring element allows the plunger to be moved quickly with a selected force, while minimizing electrical power needed to keep the plunger in one of the positions thereby reducing heat generated by the solenoid valve.

In some embodiments, the plunger is moved from one position to another by changing the polarity of the solenoid coil twice. The polarity of the solenoid coil is changed once to initiate movement of the plunger and then changed again when the center of the permanent magnet passes the center of the solenoid coil to ensure that the plunger continues to move in the same direction, as is described in more detail with reference to disclosed embodiments.

In some embodiments, a sensor (e.g., pressure or flow sensor) connected to or coupled with the aspiration channel provides a signal indicative of a fluid metric (e.g., pressure level) in the aspiration channel. A controller controls the solenoid coil to selectively open or close the aspiration and bypass channel based on the fluid metric (e.g., pressure level). In some embodiments, when the controller detects a rate of change in the fluid metric (e.g., pressure level) in the aspiration channel passing (e.g., exceeding) a given rate of change, which is indicative of an occlusion breaking, the controller quickly (for example, in 10 milliseconds or less) activates the solenoid valve to: close the aspiration channel thereby isolating the eye from the vacuum in the second section of the aspiration channel and in the aspiration tubing line until the pressure in the aspiration channel and/or aspiration line returns to a desired and/or safe pressure; and open the bypass channel so that pressure in the second section of the aspiration channel may be changed, in a non-time critical manner, via irrigation fluid from the irrigation channel entering the aspiration channel via the bypass channel as mentioned previously. In some embodiments, once the pressure in the second section of the aspiration channel passes (e.g., exceeds) a given value (e.g., given pressure level), the controller activates the solenoid valve to reopen the aspiration channel without causing a vacuum surge, which could damage the eye.

In some embodiments, in addition to being linear, the solenoid valve is small and may be produced at low-cost thereby allowing the valve to be disposed of after use. Therefore, in some embodiments, the valve does not need to withstand repeated sterilization. The valve may be housed in a cartridge which may be reversibly coupled with the phacoemulsification probe and aspiration and irrigation tubes. The cartridge may then be removed from the probe and tubes after use for cleaning or disposal.

In some embodiments, sensors (e.g., a pressure sensor for the aspiration channel and a pressure sensor for the irrigation channel) may be included in the cartridge. Including the sensors in the cartridge may provide higher sensitivity to local changes in fluid dynamics and provide a higher degree of control of the pressure in the eye.

In some embodiments, the controller is also included in the cartridge. Including the controller in the cartridge may allow the controller to be configured for the calibration of the solenoid valve. Additionally, or alternatively, including the controller in the cartridge allows the controller to be close to the sensor or sensors which may be providing analog signals that could degrade if the signals needed to travel over a cable to a remote console in which the controller may otherwise be installed.

### System Description

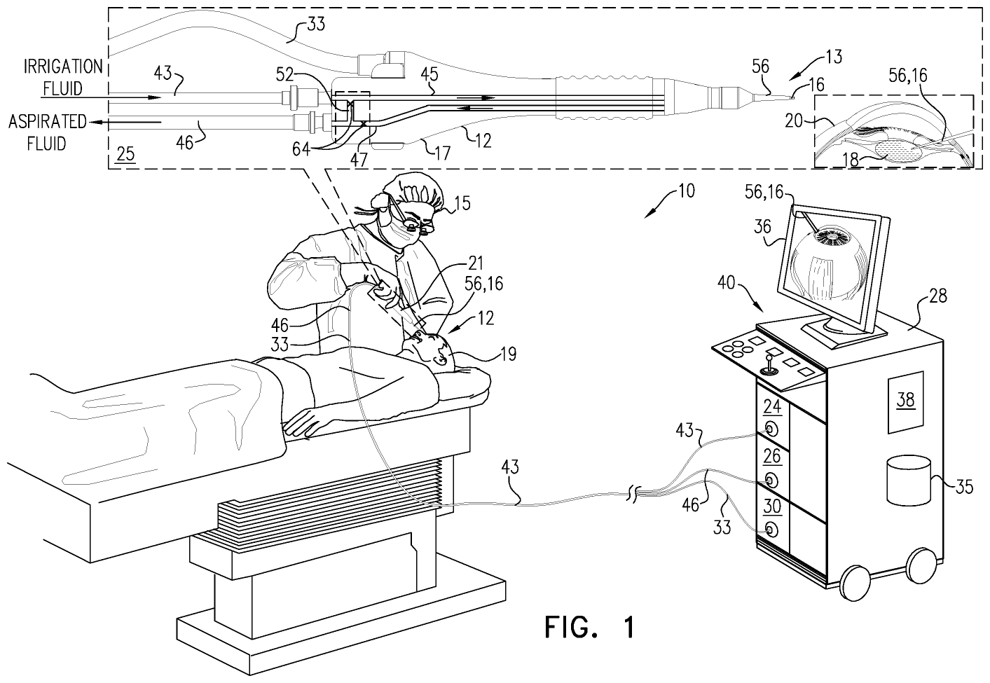

Reference is now made to FIG. 1 is a partly pictorial, partly block diagram view of a phacoemulsification system 10 constructed and operative in accordance with an embodiment of the present invention.

The phacoemulsification system 10 comprises a phacoemulsification probe 12 (e.g., handpiece). In some embodiments, the phacoemulsification probe 12 may be replaced by any suitable medical tool. As seen in the pictorial view of phacoemulsification system 10, and in inset 25, phacoemulsification probe 12 comprises: a distal end 13 including a needle 16; a probe body 17; and a coaxial irrigation sleeve 56 that at least partially surrounds needle 16 and creates a fluid pathway between the external wall of the needle and the internal wall of the irrigation sleeve, where needle 16 is hollow to provide an aspiration channel. Moreover, irrigation sleeve 56 may have one or more side ports at, or near, the distal end to allow irrigation fluid to flow towards the distal end of the phacoemulsification probe 12 through the fluid pathway and out of the port(s).

Needle 16 is configured for insertion into a lens capsule 18 of an eye 20 of a patient 19 by a physician 15 to remove a cataract. While the needle 16 (and irrigation sleeve 56) are shown in inset 25 as a straight object, any suitable needle may be used with phacoemulsification probe 12, for example, a curved or bent tip needle commercially available from Johnson & Johnson Surgical Vision, Inc., Santa Ana, Calif., USA.

In the embodiment of FIG. 1, during the phacoemulsification procedure, a pumping sub-system 24 comprised in a console 28 pumps irrigation fluid from an irrigation reservoir (not shown) to the irrigation sleeve 56 to irrigate the eye 20. The irrigation fluid is pumped via an irrigation tubing line 43 running from the console 28 to an irrigation channel 45 of probe 12. The distal end 13 of the irrigation channel 45 includes the fluid pathway in the irrigation sleeve 56. The irrigation channel 45 is configured to convey the irrigation fluid to the distal end 13. The irrigation tubing line 43 is typically flexible and may be prone to collapsing during an occlusion of the needle 16. In another embodiment, the pumping sub-system 24 may be coupled or replaced with a gravity fed irrigation source such as a BSS bottle/bag.

The phacoemulsification probe 12 includes an aspiration channel 47 configured to convey eye fluid and waste matter (e.g., emulsified parts of the cataract) away from the distal end 13. The aspiration channel 47 extends from the hollow of needle 16 through the phacoemulsification probe 12, and then via an aspiration tubing line 46 to a collection receptacle (not shown) in the console 28. The phacoemulsification system 10 includes a pumping sub-system 26 disposed in the console 28 and configured to be coupled to the aspiration tubing line 46 and pump the eye fluid and waste matter away from the distal end 13 via the aspiration channel 47 and aspiration tubing line 46.

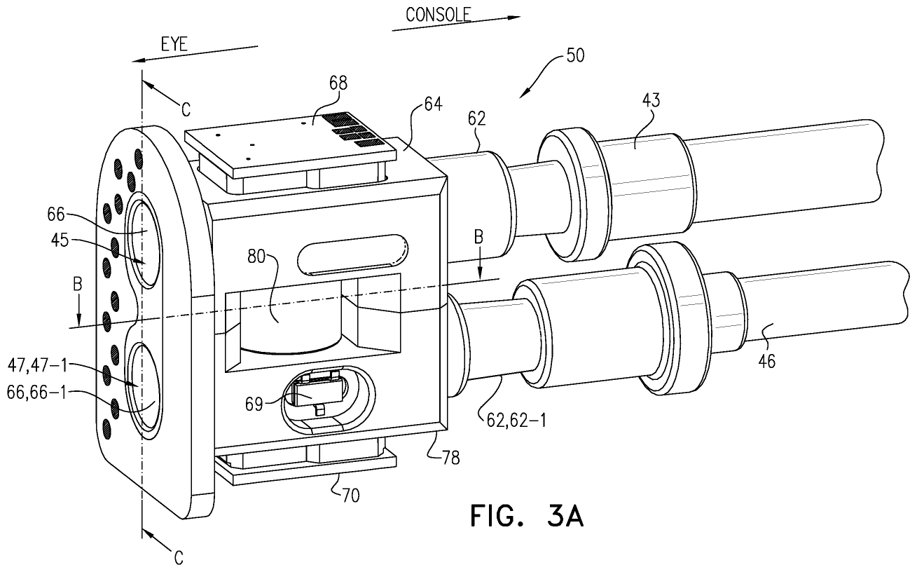

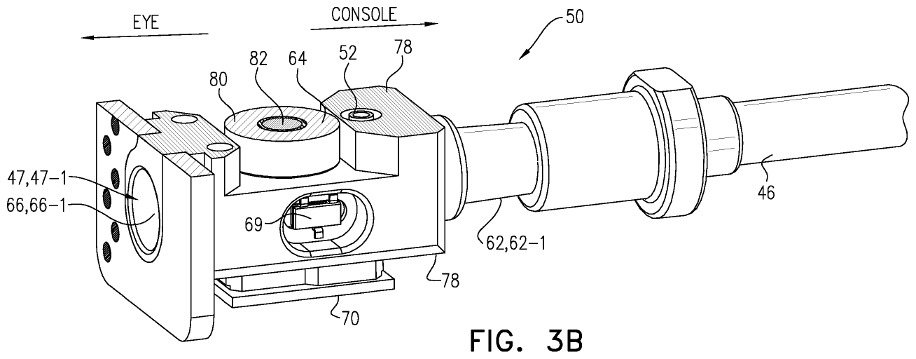

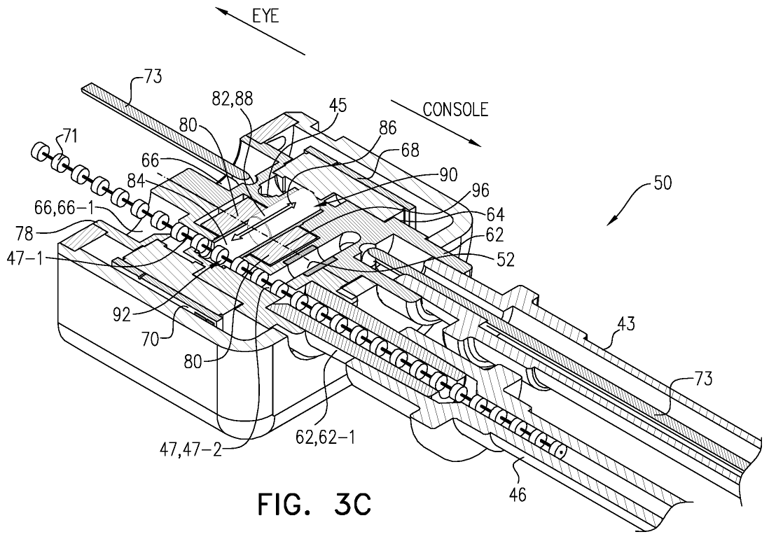

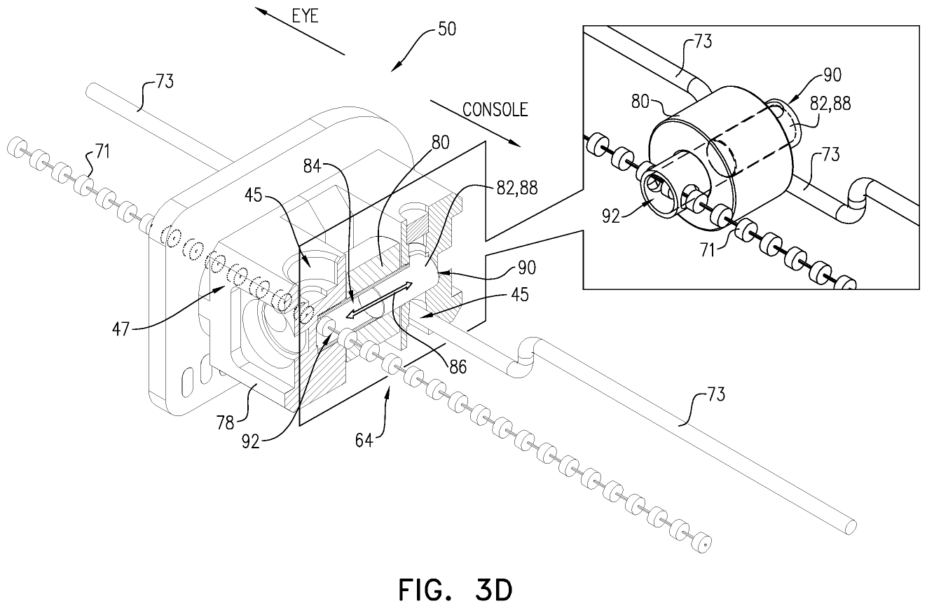

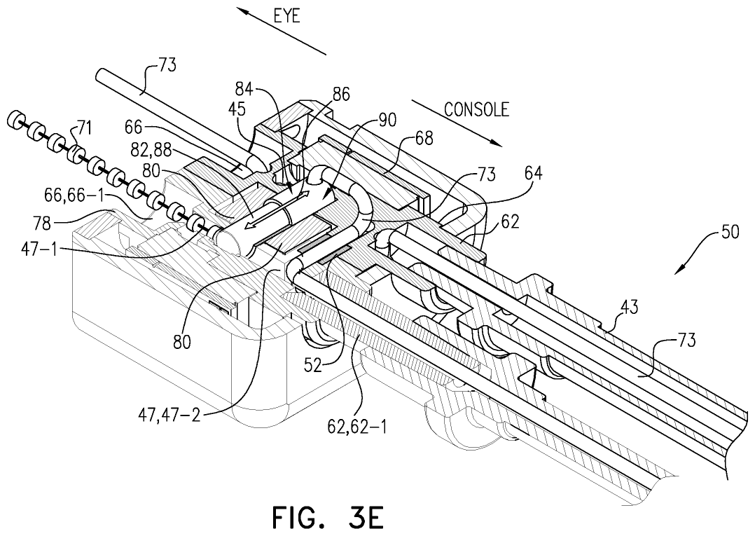

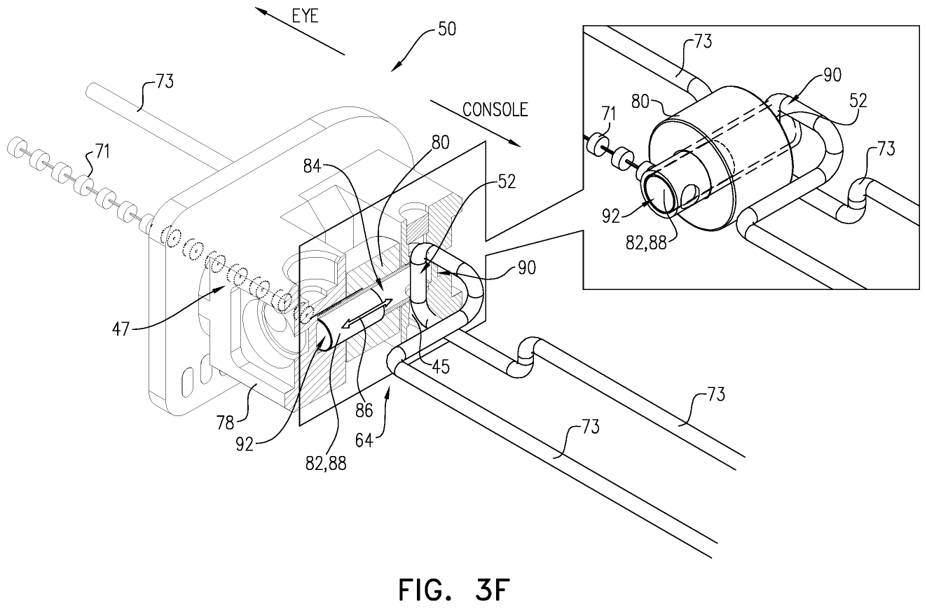

The phacoemulsification probe 12 includes a solenoid valve 64. The solenoid valve 64 is described in more detail with reference to FIGS. 3A-6. The phacoemulsification probe 12 includes a bypass channel 52 connected to the irrigation channel 45 and the aspiration channel 47, as described in more detail with reference to FIG. 3C.

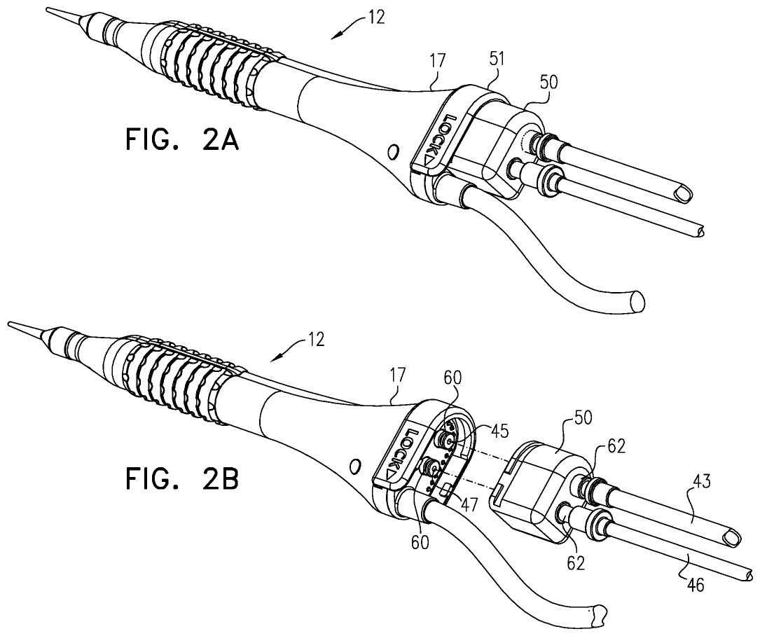

In some embodiments, the system 10 includes a fluid dynamics cartridge 50 (which may be removable), which includes the solenoid valve 64, the bypass channel 52, and sensors, described in more detail with reference to FIGS. 2A-6. Part of the irrigation channel 45 and the aspiration channel 47 may be disposed in the probe body 17 and part disposed in the cartridge 50.

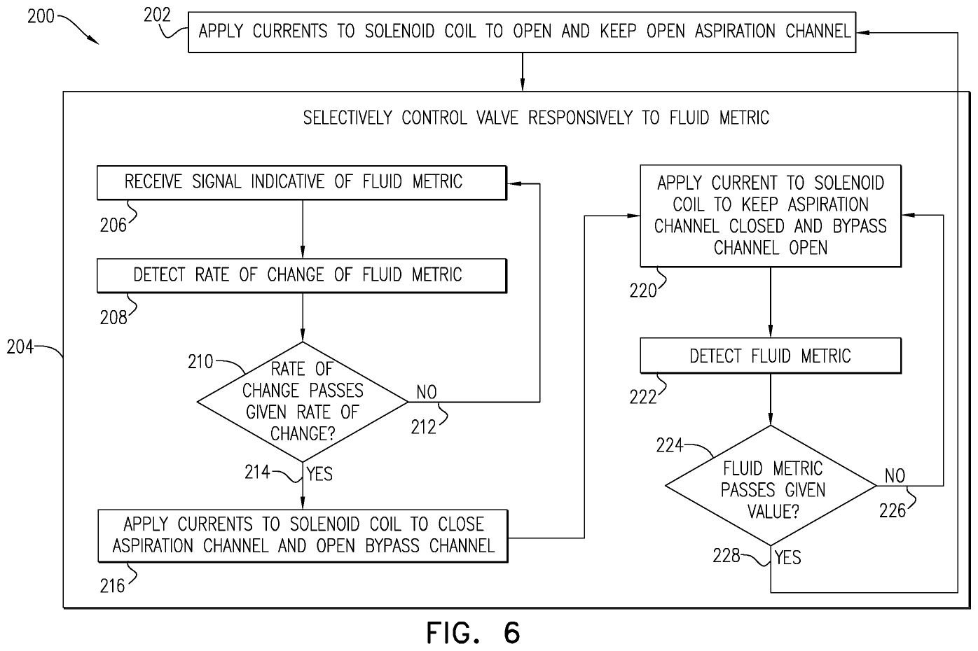

Phacoemulsification probe 12 includes other elements, such as a piezoelectric crystal (not shown) coupled to a horn (not shown) to drive vibration of needle 16. The piezoelectric crystal is configured to vibrate needle 16 in a resonant vibration mode. The vibration of needle 16 is used to break a cataract into small pieces during a phacoemulsification procedure. Console 28 comprises a piezoelectric drive module 30, coupled with the piezoelectric crystal, using electrical wiring running in a cable 33. Drive module 30 is controlled by a controller 38 and conveys processor-controlled driving signals via cable 33 to, for example, maintain needle 16 at maximal vibration amplitude. The drive module may be realized in hardware or software, for example, in a proportional-integral-derivative (PID) control architecture. The controller 38 may also be configured to receive signals from sensors in the phacoemulsification probe 12 and control one or more valves to regulate the flow of fluid in the irrigation channel 45, the aspiration channel 47, and/or the bypass channel 52 as described in more detail with reference to FIG. 6. In some embodiments, at least some of the functionality of the controller 38 may be implemented using a controller disposed in the phacoemulsification probe 12 (e.g., in the cartridge 50).

Controller 38 may receive user-based commands via a user interface 40, which may include setting a vibration mode and/or frequency of the piezoelectric crystal, and setting or adjusting an irrigation and/or aspiration rate of the pumping sub-systems 24, 26. In some embodiments, user interface 40 and a display 36 may be combined as a single touch screen graphical user interface. In some embodiments, the physician 15 uses a foot pedal (not shown) as a means of control. Additionally, or alternatively, controller 38 may receive the user-based commands from controls located in a handle 21 of probe 12.

Some or all of the functions of controller 38 may be combined in a single physical component or, alternatively, implemented using multiple physical components. These physical components may comprise hard-wired or programmable devices, or a combination of the two. In some embodiments, at least some of the functions of controller 38 may be carried out by suitable software stored in a memory 35 (as shown in FIG. 1). This software may be downloaded to a device in electronic form, over a network, for example. Alternatively, or additionally, the software may be stored in tangible, non-transitory computer-readable storage media, such as optical, magnetic, or electronic memory.

The system shown in FIG. 1 may include further elements which are omitted for clarity of presentation. For example, physician 15 typically performs the procedure using a stereo microscope or magnifying glasses, neither of which are shown. Physician 15 may use other surgical tools in addition to probe 12, which are also not shown in order to maintain clarity and simplicity of presentation.

Reference is now made to FIGS. 2A-B, which are views of the phacoemulsification probe 12 for use with the system 10 of FIG. 1. FIG. 2A shows the cartridge 50, which is configured to be reversibly attached (using a clip 51) to the probe body 17 of the phacoemulsification probe 12. FIG. 2B shows the cartridge 50 detached from the probe body 17. FIG. 2B shows ports 60 of the irrigation channel 45 and the aspiration channel 47 on the probe body 17 for connecting with corresponding ports (not shown in FIG. 2B, but shown in FIG. 3A) of the cartridge 50. FIG. 2B also shows irrigation tubing line 43 and aspiration tubing line 46 connected to ports 62 of the cartridge 50.

Reference is now made to FIGS. 3A-C. FIG. 3A is a schematic view of an interior of a fluid dynamics cartridge 50 for use in the phacoemulsification probe 12 of FIGS. 2A-B. FIG. 3B is a cross-section of the fluid dynamics cartridge 50 through line B:B of FIG. 3A. FIG. 3C is a cross-section of the fluid dynamics cartridge 50 through line C:C of FIG. 3A during normal operation (i.e., with the aspiration channel 47 open and the bypass channel 52 closed).

The phacoemulsification probe 12 may include sensors 68, and 70 (which may be pressure sensors), and a solenoid valve 64. In some embodiments, the cartridge 50 includes: the solenoid valve 64, which includes ports 62 for connection to the irrigation tubing line 43 and aspiration tubing line 46, ports 66 for connection to the ports 60 (FIG. 2B), and sections of the irrigation channel 45 and aspiration channel 47; the bypass channel 52; the pressure sensor 68 connected to the irrigation channel 45; and the pressure sensor 70 connected to aspiration channel 47 (as shown in FIG. 3C). The sensor 68 and the sensor 70 are configured to provide respective signals indicative of respective fluid metrics (e.g., pressure levels) in the irrigation channel 45 and in (a section 47-1 and/or a section 47-2 (FIG. 3C)) of the aspiration channel 47. FIG. 3C shows that the sensor 70 is sensing pressure in the section 47-1. Alternatively, or additionally, the sensor 70 may be configured to sense the pressure in section 47-2.

Including the sensors 68, 70 in the cartridge 50 may provide higher sensitivity to local changes in fluid dynamics and provide a higher degree of control of the pressure in the eye.

The cartridge 50 is compact and may be any suitable size. In some embodiments, the cartridge 50 may fit into a cube of 2.5 cm sides.

The solenoid valve 64 includes an inlet port 66-1 and an outlet port 62-1. The aspiration channel 47 includes: section 47-1 connected to the inlet port 66-1 and the distal end 13 (FIG. 1); and section 47-2 connected to the outlet port 62-1 (as shown in FIG. 3C). The solenoid valve 64 is configured to selectively control fluid connectivity in the aspiration channel 47 between the inlet port 66-1 and the outlet port 62-1 and in the bypass channel 52. The section 47-2 is configured to be coupled to the pumping sub-system 26 (FIG. 1) via the aspiration tubing line 46 and the outlet port 62-1.

The controller 38 is configured to selectively control the solenoid valve 64, responsively to the fluid metric (e.g., pressure level) in the section 47-1 and/or the section 47-2 of the aspiration channel 47, as described in more detail to FIG. 6. It should be noted that when the aspiration channel 47 is closed, the sensor 70 is configured to sense a fluid metric (e.g., pressure level) in the section 47-1 as shown in FIG. 3C. In some embodiments, when the aspiration channel 47 is closed, the sensor 70 is configured to sense a fluid metric (e.g., pressure level) in the section 47-2 of the aspiration channel 47.

The bypass channel 52 is connected to the irrigation channel 45 and the section 47-2 of the aspiration channel 47. The bypass channel 52 is configured to allow a portion of the irrigation fluid in the irrigation channel 45 to enter the section 47-2 of the aspiration channel 47 when a plunger 82 is in a position 92, as described in more detail below with reference to FIGS. 3E-F, and 6.

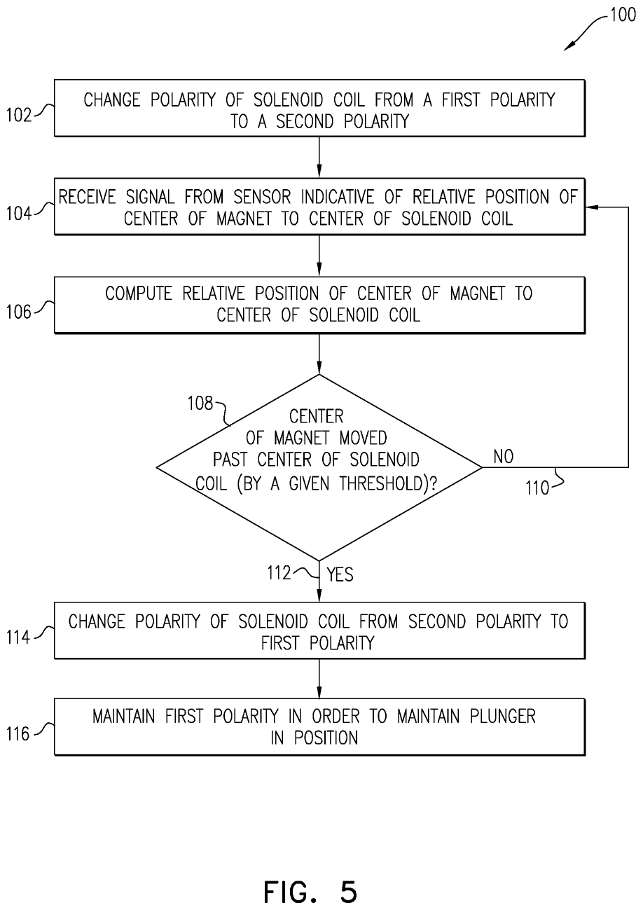

The solenoid valve 64 includes a sensor 69 (shown in FIGS. 3A and 3B). The sensor 69 may comprise a proximity sensor, for example, a Hall-effect sensor, described in more detail with reference to FIG. 5.

The solenoid valve 64 includes a valve body 78, a solenoid coil 80, and the plunger 82. The solenoid valve 64 and its operation is now described in more detail.

Reference is now made to FIGS. 3C-F. FIG. 3D is a schematic view illustrating fluid flow through the fluid dynamics cartridge 50 of FIG. 3A during normal operation. FIG. 3E is a cross-section of the fluid dynamics cartridge 50 through line C:C of FIG. 3A during occlusion release. FIG. 3F is a schematic view illustrating fluid flow through the fluid dynamics cartridge 50 of FIG. 3A during occlusion release.

The valve body 78 includes the ports 62, the ports 66, a valve cavity 84 having a direction of elongation 86 and configured to provide fluid connectivity between respective ones of the ports 62, 66 (e.g., between the inlet port 66-1 and outlet port 62-1 and in the bypass channel 52). The solenoid coil 80 is disposed in the valve body 78 around valve cavity 84. The plunger 82 includes a permanent magnet 88. The permanent magnet 88 may comprise all of, or only part of, the plunger 82. For example, the plunger 82 may include the permanent magnet 88 coated or covered with a material of low friction. The plunger 82 is configured to move back-and-forth along the direction of elongation 86 between a position 90 and position 92 in the valve cavity 84 selectively: opening the aspiration channel 47 and closing the bypass channel 52 when the plunger 82 is in the position 90 (as shown in FIGS. 3C and D); and closing the aspiration channel 47 and opening the bypass channel 52 when the plunger 82 is in the position 92 (as shown in FIGS. 3E and F). The plunger 82 may have any suitable size, for example, a length in the range of 3 mm to 2 cm (e.g., 6 mm) and a diameter in the range of 1 mm to 1 cm (e.g., 3 mm). The position 90 and the position 92 are on either side of a center 96 (FIG. 3C) of the solenoid coil 80 with respect to the direction of elongation 86.

In particular, the controller 38 is configured to selectively control the solenoid coil 80 to selectively: (a) move the plunger 82 to the position 90 to open the aspiration channel 47 allowing aspiration of eye fluid and waste matter 71 away from the distal end 13 (via the aspiration channel 47 and the aspiration tubing line 46) and close the bypass channel 52 (as shown in FIG. 3C); and (b) move the plunger 82 to the position 92 to close the aspiration channel 47 (between sections 47-1 and 47-2) and open the bypass channel 52 allowing a portion of irrigation fluid 73 in the irrigation channel 45 to enter the aspiration channel 47 reducing a vacuum in the section 47-2 of the aspiration channel 47 and in the aspiration tubing line 46 (as shown in FIGS. 3E-F). FIGS. 3E-F show that the flow of the eye fluid and waste matter 71 has been stopped by the plunger 82. The control of the solenoid coil 80 is described below in more detail with reference to FIGS. 5-6.

In FIGS. 3D and 3F the irrigation fluid 73 is seen diverting around the back of the solenoid coil 80 through channels (forming the irrigation channel 45) within the valve body 78. The diversion of the irrigation fluid 73 in the channels around the sides of the solenoid coil 80 allows the solenoid valve 64 to be more compact. Similarly, in FIGS. 3E and 3F, the irrigation fluid 73 is seen taking a number of turns through channel (forming the bypass channel 52) within the valve body 78 from the irrigation channel 45 to the section 47-2 of the aspiration channel 47 in order to allow the solenoid valve 64 to be more compact.

The plunger 82 does not have a fixed rest position in the valve cavity 84. Even though in some orientations the plunger 82 may fall in one of the positions 90, 92 due to gravity, if the solenoid valve 64 is orientated differently the plunger 84 may fall to a different position. The plunger 82 does not include a restoring element (e.g., spring) configured to restore the plunger 82 to a fixed rest position. The plunger will not always remain in the position 92 or position 90 (e.g., if the orientation of the phacoemulsification probe 12 is changed) without applying current to the solenoid coil 80. In other words, for the solenoid valve 64 to function correctly, a current is applied to the solenoid coil 80 whether the plunger 82 of the solenoid valve 64 is to remain in position 90 or in position 92. The plunger 82 will remain in the position 90 or the position 92 upon application of current to the solenoid coil 80.

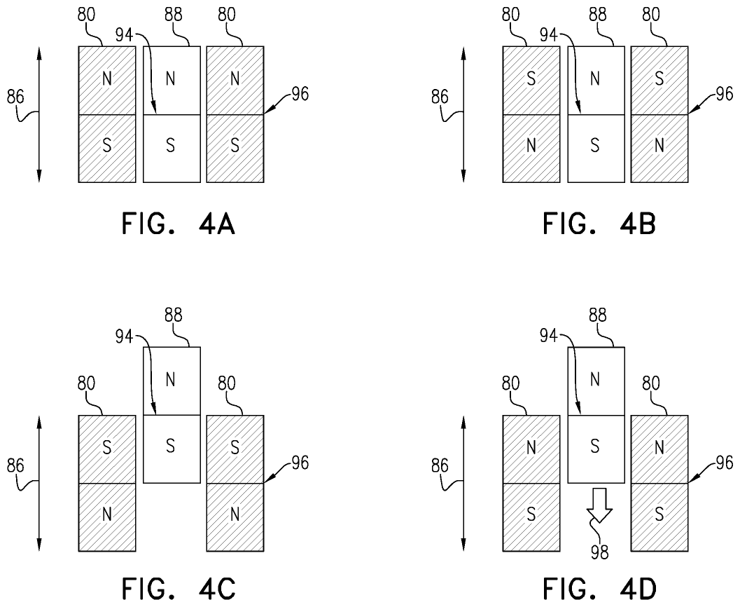

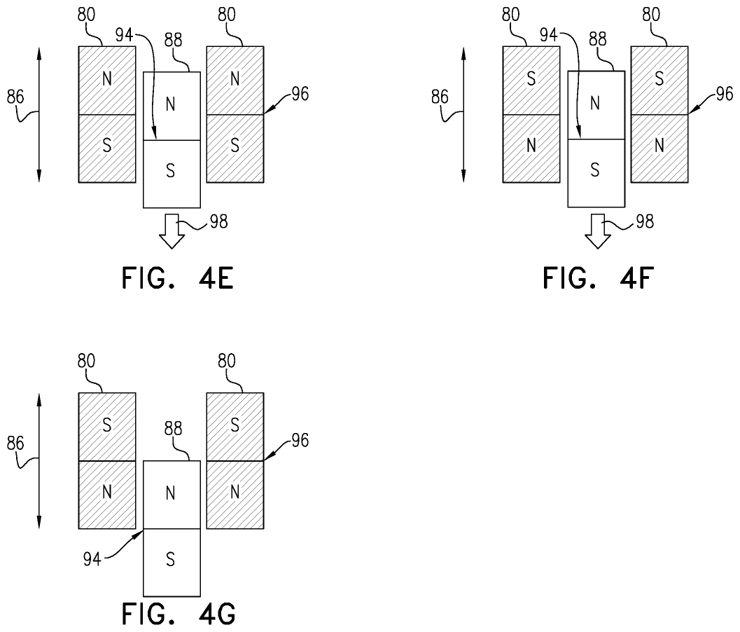

Reference is now made to FIGS. 4A-G, which are schematic views illustrating movement of the permanent magnet 88 in the solenoid coil 80 coil for use in the fluid dynamics cartridge 50 of FIG. 3A.

The permanent magnet 88 has a center 94 with respect to the direction of elongation 86. The solenoid coil 80 has a center 96 with respect to the direction of elongation 86.

In the configuration of FIG. 4A, the polarity of the solenoid coil 80 is aligned with the polarity of the permanent magnet 88. In other words, the north poles of the permanent magnet 88 are facing the same direction as the north poles of the solenoid coil 80 with respect to the direction of elongation 86. In such a configuration, if the center 94 of the permanent magnet 88 is moved a little away from the center 96 of the solenoid coil 80, in the direction of elongation 86, the permanent magnet 88 will oscillate around the center 96 of the solenoid coil 80 along the direction of elongation 86 until the permanent magnet 88 settles so that the center 94 of the permanent magnet 88 is aligned with the center 96 of the solenoid coil 80 with respect to the direction of elongation 86. The permanent magnet 88 therefore rests in a stable position with respect to the solenoid coil 80.

In the configuration of FIG. 4B, the polarity of the solenoid coil 80 is aligned in the opposite direction to the polarity of the permanent magnet 88. In other words, the north poles of the permanent magnet 88 are facing the opposite direction to the north poles of the solenoid coil 80 with respect to the direction of elongation 86. In such a configuration, if the center 94 of the permanent magnet 88 is moved a little away from the center 96 of the solenoid coil 80, with respect to the direction of elongation 86, the permanent magnet 88 will continue to move in that direction. The permanent magnet 88 in FIG. 4B is therefore in an unstable position with respect to the solenoid coil 80.

Assuming the permanent magnet 88 is at rest as shown in FIG. 4C, the permanent magnet 88 may be set into motion in a direction 98 (with the center 94 of the permanent magnet 88 moving towards the center 96 of the solenoid coil 80) by changing the polarity of the solenoid coil 80 as shown in FIG. 4D.

When the center 94 of the permanent magnet 88 passes the center 96 of the solenoid coil 80, as shown in FIG. 4E, the polarity of the solenoid coil 80 is changed again as shown in FIG. 4F in order for the permanent magnet 88 to continue moving in the direction 98. If the polarity of the permanent magnet 88 is not changed at that point, the center 94 of the permanent magnet 88 may just oscillate around the center 96 of the solenoid coil 80 until the permanent magnet 88 comes to rest. The permanent magnet 88 keeps moving in the direction 98 and settles at its final position as shown in FIG. 4G. In order for the permanent magnet 88 to remain in the position shown in FIG. 4G, the controller 38 is configured to keep applying a current to the solenoid coil 80 so that the polarity of the solenoid coil 80 is as shown in FIG. 4G.

In order to move the permanent magnet 88 back to the other side of the solenoid coil 80, polarity of the solenoid coil 80 is changed twice as described above.

Reference is now made to FIG. 5, which is a flowchart 100 including steps in a method of operation of the solenoid coil 80 of FIGS. 4A-G. Reference is also made to FIGS. 4A-G.

In response to a decision to move the plunger 82 (including the permanent magnet 88) from the position 90 to the position 92 or from the position 92 to the position 90, the controller 38 is configured to change the polarity of the solenoid coil 80 from a first polarity (block 102) (e.g., the current polarity of the solenoid coil 80) to a second polarity.

The sensor 69 (e.g., a Hall-effect sensor) (FIGS. 3A-B) is configured to provide a signal responsively to a relative position of the center 94 of the magnet 88 to the center 96 of the solenoid coil 80 with respect to the direction of elongation 86. The controller 38 is configured to receive the signal from the sensor 69 (block 104), the signal being indicative of the relative position of the center 94 of the permanent magnet 88 to the center 96 of the solenoid coil 80. The controller 38 is configured to compute the relative position of the center 94 of the permanent magnet 88 to the center 96 of the solenoid coil 80 (block 106), responsively to the received signal. At a decision block 108, the controller 38 is configured to determine if the center 94 of the permanent magnet 88 has moved past the center 96 of the solenoid coil 80 (optionally by a given threshold), with respect to the direction of elongation 86. If the center 94 of the permanent magnet 88 has not moved past the center 96 of the solenoid coil 80 (by a given threshold) (branch 110), processing returns to the step of block 104. If the center 94 of the permanent magnet 88 has moved past the center 96 of the solenoid coil 80 (by a given threshold) (branch 112), the controller 38 is configured to change the polarity from the second polarity to the first polarity (block 114) responsively to the provided signal from the sensor 69. Therefore, the controller 38 is configured to change the polarity from the second polarity to the first polarity responsively to the center 94 of the magnet 88 passing the center 96 of the solenoid coil 80 with respect to the direction of elongation 86. In some embodiments, if the plunger 82 has enough momentum to pass the center even though the polarity is changed prior to the permanent magnet 88 passing the center 96 of the solenoid coil 80, then the polarity could even be changed prior to the permanent magnet 88 passing the center 96 of the solenoid coil 80. Therefore, in order to move the plunger 82 from the position 92 to the position 90 (or vice-versa), the controller 38 is configured to change the polarity of the solenoid coil 80 from the first polarity to the second polarity and then back to the first polarity. The controller 38 is configured to control the solenoid coil 80 to maintain the first polarity in order to maintain the plunger 82 in position (block 116) (e.g., in position 90 or position 92).

Reference is now made to FIG. 6, which is a flowchart 200 including steps in a method of operation of system 10 of FIG. 1. Reference is also made to FIG. 3C. The controller 38 is configured to apply currents to the solenoid coil 80 to open (and keep open) and the aspiration channel 47 (block 202) as described with reference to FIG. 5.

The controller 38 is configured to selectively control the solenoid valve 64 responsively to the fluid metric (e.g., pressure level) in the aspiration channel 47 (block 204). The step of block 204 is now described in more detail with reference to sub-steps of blocks 206-224.

The controller 38 is configured to receive a signal indicative of the fluid metric (e.g., pressure level) in the section 47-1 and/or the section 47-2 of the aspiration channel 47 from the pressure sensor 70 (block 206). The controller 38 is configured to detect a rate of change of the fluid metric (e.g., pressure level) in the section 47-1 and/or the section 47-2 of the aspiration channel 47 responsively to the received signal (block 208). At a decision block 210, the controller 38 is configured to determine if the rate of change passes (e.g., exceeds) a given rate of change. If the rate of change does not pass (e.g., exceed) the given rate of change (branch 212), the method returns to the sub-step of block 206. If the rate of change passes (e.g., exceeds) the given rate of change (branch 214), the controller 38 is configured to apply currents to the solenoid coil 80 (i.e., by changing the polarity of the solenoid coil 80 twice as described above with reference to FIG. 5) to move the plunger 82 to position 92 to close the aspiration channel 47 and open the bypass channel 52 (FIG. 1) (block 216) protecting the eye from a vacuum surge. Therefore, the controller 38 is configured to control the solenoid coil 80 to selectively move the plunger 82 between the position 90 and the position 92 responsively to the signal provided by the sensor 70.

The controller 38 is configured to continue to apply a current to the solenoid coil 80 to keep the aspiration channel 47 closed and the bypass channel 52 open (block 220). In this manner the vacuum in the aspiration tubing line 46 and the section 47-2 of the aspiration channel 47 between the solenoid valve 64 and the aspiration tubing line 46 is allowed to reduce by allowing a portion of the irrigation fluid in the irrigation channel 45 to enter the section 47-2 of the aspiration channel 47 via the bypass channel 52. The entry of irrigation fluid reduces the vacuum and increases the fluid metric (e.g., pressure level) in the section 47-2 of the aspiration channel 47 and the aspiration tubing line 46.

In embodiments where the sensor 70 is configured to sense the pressure in the section 47-2 of the aspiration channel 47 (even when the aspiration channel 47 is closed), the controller 38 is configured to detect the fluid metric (e.g., pressure level) in the section 47-2 of the aspiration channel 47 responsively to signal received from the pressure sensor 70 (block 222). At a decision block 224, the controller 38 is configured to determine if the fluid metric (e.g., pressure level) in the section 47-2 of the aspiration channel 47 passes (e.g., exceeds) a given value (e.g., given pressure level). If fluid metric (e.g., pressure level) does not pass (e.g., exceed) the given value (e.g., given pressure level) (branch 226), the sub-step of block 220 is repeated. If the fluid metric (e.g., pressure level) in the section 47-2 of the aspiration channel 47 passes (e.g., exceeds) the given value (e.g., given pressure level) (branch 228), the step of block 202 is repeated in which the controller 38 is configured to control the solenoid coil 80 to move the plunger 82 to position 90 in which the aspiration channel 47 is opened and the bypass channel 52 is closed.

As used herein, the terms “about” or “approximately” for any numerical values or ranges indicate a suitable dimensional tolerance that allows the part or collection of components to function for its intended purpose as described herein. More specifically, “about” or “approximately” may refer to the range of values ±20% of the recited value, e.g., “about 90%” may refer to the range of values from 72% to 108%.

Various features of the invention which are, for clarity, described in the contexts of separate embodiments may also be provided in combination in a single embodiment. Conversely, various features of the invention which are, for brevity, described in the context of a single embodiment may also be provided separately or in any suitable sub-combination.

The embodiments described above are cited by way of example, and the present invention is not limited by what has been particularly shown and described hereinabove. Rather the scope of the invention includes both combinations and sub-combinations of the various features described hereinabove, as well as variations and modifications thereof which would occur to persons skilled in the art upon reading the foregoing description and which are not disclosed in the prior art.

What is claimed is:

1. A fluid dynamics system, comprising:
a solenoid valve comprising:
a valve body comprising a valve cavity having a direction of elongation, a first channel, and a second channel;
a solenoid coil disposed in the valve body around the valve cavity; and
a plunger comprising a magnetic element and configured to move back-and-forth along the direction of elongation between a first position and a second position in the valve cavity, selectively: opening the first channel and closing the second channel when the plunger is in the first position; and closing the first channel and opening the second channel when the plunger is in the second position; and
a controller configured to control the solenoid coil to selectively move the plunger between the first position and the second position, and to selectively maintain the plunger in the first position and the second position.

2. The system according to claim 1, wherein the plunger does not have a fixed rest position in the valve cavity.

3. The system according to claim 1, wherein the plunger does not include a restoring element configured to restore the plunger to a fixed rest position.

4. The system according to claim 1, wherein the plunger will not remain in the first position and the second position without applying a current to the solenoid coil.

5. The system according to claim 1, wherein the plunger will remain in the first position or the second position upon application of a current to the solenoid coil.

6. The system according to claim 1, wherein:
the solenoid coil has a center with respect to the direction of elongation;
the first position and the second position are on either side of the center of the solenoid coil with respect to the direction of elongation; and
the controller is configured to:
change a polarity of the solenoid coil from a first polarity to a second polarity and then back to the first polarity to move the plunger from the first position to the second position; and
change the polarity of the solenoid coil from the first polarity to the second polarity and then back to the first polarity to move the plunger from the second position to the first position.

7. The system according to claim 6, wherein the controller is configured to control the solenoid coil to maintain the first polarity in order to maintain the plunger in position.

8. The system according to claim 6, wherein:
the magnetic element has a center with respect to the direction of elongation; and
the controller is configured to change the polarity from the second polarity to the first polarity responsively to the center of the magnetic element passing the center of the solenoid coil with respect to the direction of elongation.

9. The system according to claim 8, wherein:
the solenoid valve includes a sensor configured to provide a signal responsively to a relative position of the center of the magnetic element to the center of the solenoid coil with respect to the direction of elongation; and
the controller is configured to change the polarity from the second polarity to the first polarity responsively to the provided signal.

10. The system according to claim 9, wherein the sensor includes a Hall-effect sensor.

11. The system according to claim 1, further comprising a phacoemulsification probe including: a distal end comprising a needle; an irrigation channel configured to convey irrigation fluid to the distal end; an aspiration channel configured to convey eye fluid and waste matter away from the distal end, the aspiration channel comprising the first channel; and a bypass channel connected to the irrigation channel and the aspiration channel, the bypass channel comprising the second channel, and wherein the controller is configured to selectively control the solenoid coil to:
(a) move the plunger to the first position to open the first channel of the aspiration channel and close the second channel of the bypass channel allowing aspiration of the eye fluid and waste matter away from the distal end; and
(b) move the plunger to the second position to close the first channel of the aspiration channel and open the second channel of the bypass channel allowing a portion of the irrigation fluid in the irrigation channel to enter the aspiration channel reducing a vacuum in part of the aspiration channel.

12. The system according to claim 11, further comprising:
an aspiration tubing line; and
a pumping sub-system configured to be coupled to the aspiration tubing line and pump the eye fluid and waste matter away from the distal end via the aspiration tubing line and the aspiration channel, and wherein the first channel of the aspiration channel includes a first section connected to the distal end, and a second section configured to be coupled to the pumping sub-system via the aspiration tubing line, the bypass channel being configured to allow the portion of the irrigation fluid in the irrigation channel to enter the second section of the aspiration channel when the plunger is in the second position.

13. The system according to claim 12, further comprising a sensor configured to provide a signal indicative of a fluid metric in the second section of the aspiration channel, and wherein the controller is configured to control the solenoid coil to selectively move the plunger between the first position and the second position responsively to the signal.

14. The system according to claim 13, wherein the fluid metric is a pressure level,

15. The system according to claim 13, wherein the controller is configured to: detect a rate of change of the fluid metric in the second section of the aspiration channel; and control the solenoid coil to move the plunger to the second position responsively to the detected rate of change passing a given rate of change allowing the portion of the irrigation fluid in the irrigation channel to enter the second section of the aspiration channel via the bypass channel increasing the fluid metric in the second section of the aspiration channel.

16. The system according to claim 15, wherein the controller is configured to control the solenoid coil to move the plunger to the first position responsively to the fluid metric in the second section of the aspiration channel passing a given value.

17. The system according to claim 13, wherein the phacoemulsification probe further comprises a probe body and a fluid dynamics cartridge configured to be reversibly connected to the probe body, the fluid dynamics cartridge comprising the solenoid valve, the sensor, and the bypass channel.

18. A fluid dynamics method, comprising:
changing a polarity of a solenoid coil from a first polarity to a second polarity and then back to the first polarity to move a plunger comprising a magnetic element from a first position to a second position in a valve cavity;
changing the polarity of the solenoid coil from the first polarity to the second polarity and then back to the first polarity to move the plunger from the second position to the first position; and
controlling the solenoid coil to maintain the first polarity in order to maintain the plunger in the first position or the second position.

19. The method according to claim 18, wherein the changing the polarity includes changing the polarity of the solenoid coil from the second polarity to the first polarity responsively to a center of the magnetic element passing a center of the solenoid coil with respect to a direction of elongation of the valve cavity.

20. The method according to claim 19, further comprising providing a signal responsively to a relative position of the center of the magnetic element to the center of the solenoid coil with respect to the direction of elongation, and wherein the changing the polarity is performed responsively to the provided signal.

21. The method according to claim 20, wherein the providing is performed by a Hall-effect sensor.
